(19) United States
(12) Patent Application Publication
(10) Pub. No.: US 2015/0342683 A1
(43) Pub. Date: Dec. 3, 2015

(54) Instrument Support System for Attachment to Mayo Stand Frame

(71) Applicants: Mark P. Zoland, Scarsdale, NY (US); Joseph Iraci, New Rochelle, NY (US)

(72) Inventors: Mark P. Zoland, Scarsdale, NY (US); Joseph Iraci, New Rochelle, NY (US)

(21) Appl. No.: 14/293,658

(22) Filed: Jun. 2, 2014

(51) Int. Cl. A61B 19/02

(52) U.S. Cl. CPC A61B 19/0256; A61B 2019/0251

(57) ABSTRACT

An instrument support system includes a receptacle for a surgical instrument and a support that suspends the receptacle from a frame of a mayo stand so that the receptacle is located below the frame, at least partially within the perimeter of the frame, and preferably at an oblique angle. A receptacle includes an open distal end to permit partial passage of the instrument therethrough. The system also includes a cord holder to support the cord or other flexible lumen of the instrument outside the receptacle.

### BACKGROUND OF THE INVENTION

1. Field of the Invention

This invention relates broadly to receptacles used in operating rooms for holding or temporarily storing surgical instruments. More particularly, this invention relates to surgical instrument receptacles used in conjunction with a mayo stand.

2. State of the Art

Instruments used by surgeons during surgical procedures are commonly laid out on a moveable table called a “mayo stand”. A mayo stand includes a vertical leg, a rectangular frame horizontally cantilevered at the upper end of the leg, and a wheeled base at the lower end of the leg to provide mobility to the stand. A removable tray is seated on the frame. A typical mayo stand tray may be 13 inches wide by 19 inches long and is generally supported at its longitudinal ends on the frame.

During a surgical procedure, the tray is covered in a sterile material, and instruments to be used during surgery are placed on the tray. Then, as the instruments are required for the procedure, each is picked up from the tray by the surgeon, or an assistant hands the requested instrument to the surgeon.

Co-owned U.S. Pat. Nos. 7,980,517 and 8,453,977 describe neutral field trays for ‘sharps’ instruments that are designed to attached to the mayo frame with a hook, and to extend under the frame of the stand at an ergonomic angle. The neutral field tray is structurally and functionally adapted to act a ‘neutral field’ for sharps that are passed between a surgeon and an assistant. During a surgical procedure, each time a surgeon requires a sharps instrument, it is presented into the neutral field of the tray and then retrieved from there, eliminating hand-to-hand passage which can lead to injury. The neutral field tray is also adapted with structural features that retain the sharps end of the instrument in a manner that it is shielded from the surgeon's and assistant's hands.

### SUMMARY OF THE INVENTION

The present invention provides an instrument support system including an instrument receptacle that is adapted to receive a surgical instrument and a receptacle support that suspends the receptacle from the frame of a mayo stand so that the surgical instrument can be supported during a variety of surgical procedures. The surgical instruments used in such procedures include a proximal end defining a handle portion, and a distal end for acting on or near a patient. The receptacle is designed for instruments having a distal end of a non-sharp construct, such as an electrocautery device, a suction or aspiration device, or a visualization scope. Specifically, the surgical instruments may be powered, and include appropriate electrical cords, optical cables, suction lumens, etc., extending from their handle portion for connection to associated power sources.

The instrument receptacle includes a proximal mouth, a bottom, side walls, and a distal end opposite the mouth. The mouth is wider than the distal end such that the receptacle forms a funnel that facilitates receiving the instrument. In a receptacle of the system, the receptacle includes an opening at its distal end to receive a distal portion of the instrument therethrough. In a receptacle of the system, the bottom of the receptacle includes a slot open to the mouth to receive a handle of the instrument and retain the handle in a preferably upright orientation. The slot allows a portion of the handle to intersect a surface defined by the bottom. In a receptacle of the system, a side wall of the receptacle may be provided with a cord holder adapted to support and retain a portion of an electric power cord, vacuum lumen, or other flexible elongate structure outside the receptacle. It is preferred, though not necessary to the system, that the receptacle have an open top to facilitate construction.

In one embodiment, the receptacle support is removably attachable to the frame of a mayo stand and has a portion which can extend below the frame of the mayo stand without substantially interrupting the seating of the mayo tray on the frame of the mayo stand. In accord with one embodiment of the receptacle support, the support includes an upper hook portion, and an engagement portion that releasably engages the receptacle. The engagement portion may include arms that are adapted to extend about the side walls of the receptacle. The arms may also extend about at least a portion of the bottom of the receptacle. The arms preferably define a tapered opening therebetween that is sized to receive and retain the receptacle without permitting passage of the receptacle completely through the arms.

The engagement portion may also be provided with a cover portion which is optionally provided with a perimeter lip. When the engagement portion is coupled to the receptacle, the cover portion extends over the receptacle (i) to provide a protective cover thereover, and (ii) to increase laterally rigidity to the retained receptacle thereby reinforcing the receptacle construction. The lip provides a lateral barrier which increases laterally rigidity of the sidewalls of receptacle. These features allow the receptacle to be made of less rigid, and preferably less expensive, materials than the receptacle support. The support, e.g., at the arms, may also include a cord holder adapted to support and retain a portion of flexible cord of the instrument external of the receptacle.

The engagement portion is configured to stably receive and suspend the receptacle below the frame of the mayo stand, preferably at least partially within the footprint of the frame of the mayo stand, and preferably at an oblique angle relative to the top of the frame of the mayo stand. As such, with the receptacle so held and oriented, the receptacle is adapted to receive, retain and orient a surgical instrument for the surgeon in a convenient and accessible location during a surgical procedure.

The support may be a universal type design adapted to releasably receive instrument receptacles of various designs. In accord with one embodiment of the system, the support is constructed of materials permitting it to be a re-usable and re-sterilizable device. In an embodiment, the instrument receptacle is constructed of a different material than the support and is intended as a disposable single-use item.

A kit is also provided of a support with a plurality of receptacles of different design, each of which can be coupled relative to the support.

Additional objects and advantages will become apparent to those skilled in the art upon reference to the detailed description taken in conjunction with the provided figures.

### BRIEF DESCRIPTION OF THE DRAWINGS

Prior Art FIG. 1 is an operating table and partially exploded mayo stand and tray positioned about the operating table.

FIG. 2 is a perspective view of an embodiment of an instrument support system coupled to the frame of the mayo stand, and wherein a receptacle of the system is provided with a surgical instrument.

FIG. 3 is a perspective partial assembly view of the instrument support system, in which an engagement portion of the system is attached to the frame of the mayo stand, and the receptacle portion of the system is moved into engagement with the engagement portion.

FIG. 4 is a top view of the embodiment of the receptacle shown in FIGS. 2 and 3.

FIG. 5 is a proximal end view of the embodiment of the receptacle shown in FIGS. 2 and 3.

FIG. 6 is a side perspective view of the embodiment of the receptacle shown in FIGS. 2 and 3.

FIG. 7 is a top view of another embodiment of a receptacle.

FIG. 8 is a proximal end view of the embodiment of the receptacle shown in FIG. 7.

FIG. 9 is a side perspective view of the embodiment of the receptacle shown in FIG. 7.

### DETAILED DESCRIPTION OF THE PREFERRED EMBODIMENTS

With reference to the devices and systems described herein which are intended to be hand-operated and hand-accessed by a user, the terms “proximal” and “distal” are defined in reference to the user's hand in the assembled and in-use configuration, with the term “proximal” being closer to the user's hand, and the term “distal” being further from the user's hand, unless alternate definitions are specifically provided.

Turning now to Prior Art FIG. 1, a mayo stand 10 for supporting a surgical instrument during a variety of surgical procedures is shown. The mayo stand 10 has a base 12 with wheels 14, a vertical leg 16 extending upward from the base 12, and a generally horizontal rectangular frame 18 attached to the vertical leg 16. The frame 18 has an upper surface 20 and a lower surface 22 that together extends about a perimeter of the frame. The perimeter defines a suspended footprint of the frame. The perimeter of the frame 18 horizontally supports a mayo tray 24 (or table), which rests on the upper surface 20 of the frame 18, and is used by scrub nurses or other assistants to hold/support instruments and supplies during surgery. The mayo stand 10 and horizontal tray 24 thus provide a stable horizontal surface that is movable relative to an operating table 26 while remaining at a uniform height above the operating table 26 during a surgical procedure.

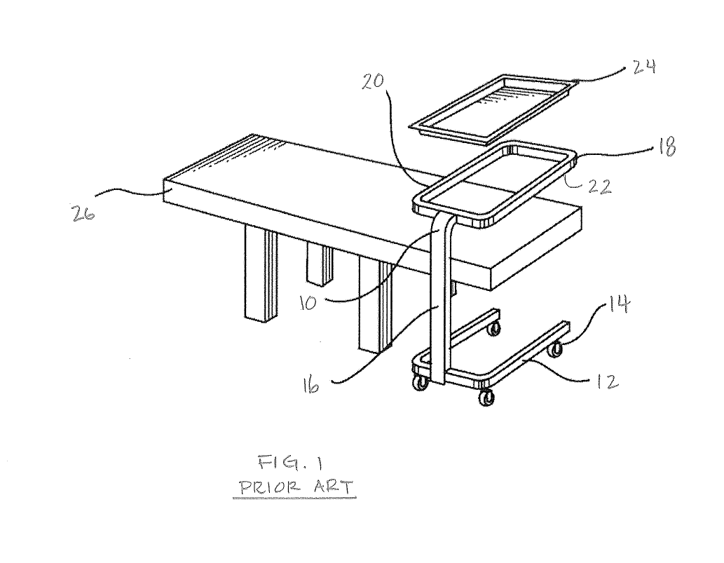

Referring now to FIG. 2, an embodiment of an instrument support system 30 for attachment to the frame 18 of the mayo stand is shown. The instrument support system 30 includes a support 32 that couples relative to the frame 18, and a receptacle 34 coupled relative to the support 32 that is adapted to receive a surgical instrument 100. The surgical instrument 100 has a proximal end 102 having a handle portion 104, optionally with finger grips 106, and a longitudinally extending distal portion 108 for acting on or near a patient. In addition, the surgical instrument 100 may be powered, and include a flexible cord 110, e.g., an electrical cord, an optical cable, a suction lumen, etc., extending from the handle portion 104 for connection to a corresponding associated power source, monitor, vacuum source, etc. While the receptacle 34 has been designed in view of the considerations of surgical procedures with instruments having a distal end portion 108 of a non-sharp construct, such as an electrocautery device, a suction or aspiration device, or a visualization scope, it may optionally be used with sharp instruments as well. The receptacle 34 is sized and adapted to preferably receive a single surgical instrument 100 within the receptacle at any one time.

In accord with one aspect of the system, the support 32 and receptacle 34 are separate components, removably couplable to each other, as clearly shown by FIGS. 2 and 3 and described herein below. FIG. 3 illustrates an embodiment in which the support 32 is a re-usable component, and the receptacle 34 is a replaceable and preferably disposable component that removably couples to the support.

Referring to FIGS. 2 through 6, the instrument receptacle 34 includes a proximal mouth 40 through which at least a portion of the instrument 100 is received, a bottom 42 that supports the instrument, side walls 44, 46 that laterally constrain the instrument, and a distal end 48 opposite the mouth 40. The receptacle is preferably laterally symmetrical about its longitudinal axis A_{L}. The mouth 40 is wider than the distal end 48 such that the receptacle has a tapered outer shape and the interior preferably assumes the shape of a funnel that facilitates receiving an instrument 100 into the mouth 40 and directing the instrument toward the distal end 48. The receptacle has an exemplar length in the range of 3 to 6 inches, and width in the range of 1.5 to 4 (at the mouth) to 1 to 2 inches (at the distal end), but other suitable lengths and widths can be provided.

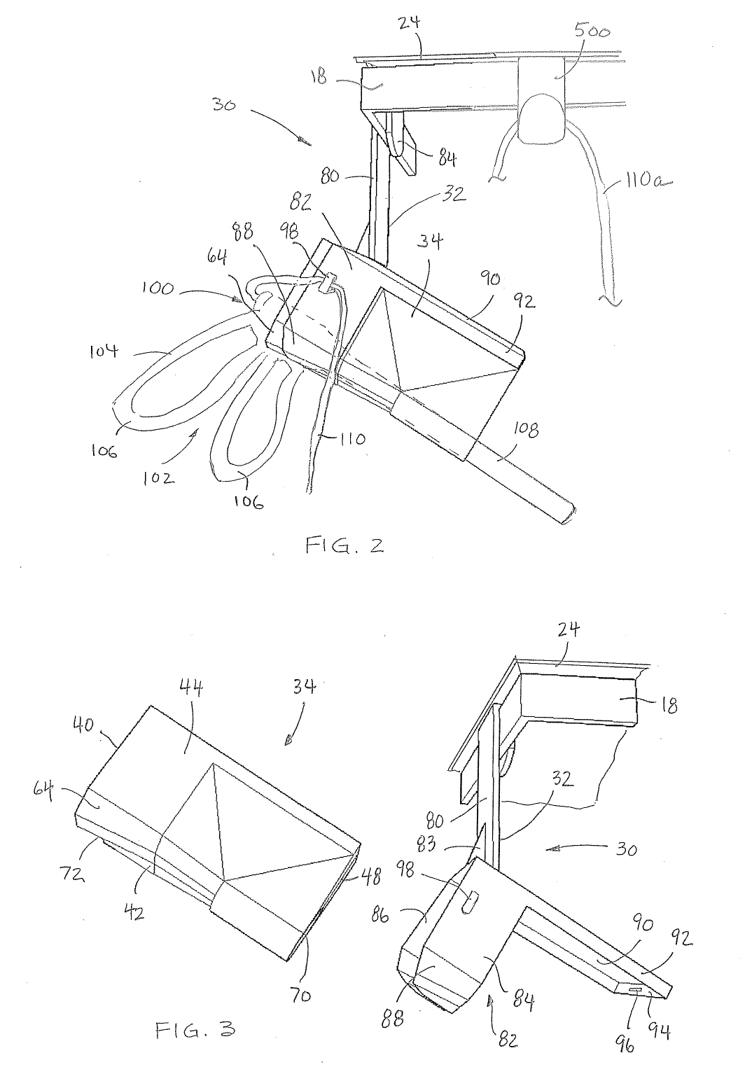

More particularly, referring specifically to FIGS. 4 through 6, an upper rim 50 is defined across the top of the side walls 44, 46 and the distal end 48. The upper rim 50 defines two straight edges 52, 54 along the side walls that are angled inward toward each other and which intersect a third straight edge 56 extending along the top of the distal end. From the upper rim 50, a proximal end portion 58 of the receptacle 34 extends downward in the shape of a shallow U, a distal end portion 60 of the receptacle tapers inward to a steeper U, and central portion 62 disposed between the proximal end and distal end portions narrows in width therebetween. The proximal end portion 58 preferably includes tapered corner wall portion 64 extending between a relatively upright portion of each side wall 44, 46 and the bottom 42 of the receptacle, and also extending from the mouth 40 to the central portion 62.

Referring to FIGS. 2 through 5, in accord with a preferred aspect of the receptacle shown, the distal end 48 is provided with an opening 70 sized to receive a longitudinally extending distal portion 108 of the instrument 100 therethrough. The opening 70 is preferably circular in shape and has a diameter sized to readily permit receiving the distal portion 108 of the instrument 100, but which also acts to stably retain the distal portion 108 of the instrument from significant lateral and upward displacement. By way of example only, the diameter of the distal end opening 70 is preferably in the range of ½ to 1 inch, but can be smaller or larger as suitable for receiving and stably retaining the distal portion of an instrument in the operating room.

Referring to FIGS. 2 through 6, in accord with another aspect of the receptacle shown, the bottom 42 of the receptacle includes a slot 72 (e.g., a recess or cutout) open at its proximal end (i.e., the mouth 40) so as to receive therein the handle 104 of the instrument 100 and retain the handle 104 in a preferably upright orientation. The slot 72 specifically allows the finger grips 106 of the handle 104 to be received and advanced into the slot 72, as shown in FIG. 2. Preferably the slot 72 is sized to retain the handle 104 from significant angular displacement relative to the bottom 42 of the receptacle 34. When the finger grips 106 of the handle 104 extend within the slot 72, the finger grips 106 intersect a surface defined by the bottom 42. Alternatively or additionally, a slot 72a (shown in broken lines) can be provided within the proximal left and/or right side walls 44, 46 of the receptacle. Such a slot 72a is well adapted to receive therein the cable extending laterally from a visualization scope and may also be suitable to receive other flexible cord structure connected to a surgical instrument.

The distal end 48 is optionally provided with a catch 74 in the form of an open channel, inward indent, tab or other structure or feature at which the receptacle 34 can be engaged, preferably from externally of the receptacle, as discussed below.

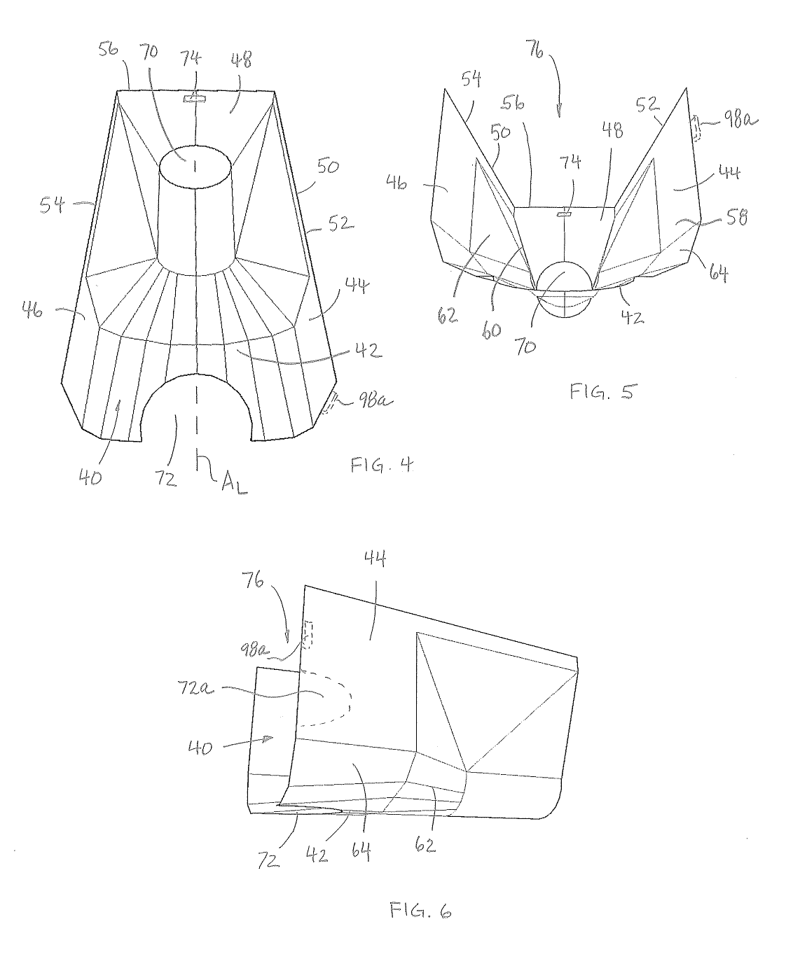

It is preferred, though not necessary to the system, that the receptacle 34 has a completely open top 76 to facilitate construction (FIGS. 5 and 6). By constructing the receptacle with a completely open top 76, molding considerations are greatly facilitated where the receptacle is manufactured from a molding process, and reduced disposable materials are required for construction processes utilizing both molded and non-molded materials. However, manufacturing considerations notwithstanding, the receptacle alternatively may be constructed with an integrated partially enclosing or fully covering top member.

Referring back to FIGS. 2 and 3, the receptacle support 32 is removably attachable to the frame 18 of the mayo stand in a manner that permits suspending the receptacle 34 below the frame. The support 32 includes an upper hook portion 80, and an engagement portion 82 that preferably releasably engages the receptacle 34. The upper hook portion 80 is sized to extend over and capture in a stable manner, e.g., by clipping over the upper surface 20 and onto the horizontal frame 18. The terminal end 84 of the hook 80 is preferably blunt, with a rounded profile. This blunt shape reduces sharp edges that can otherwise puncture a surgical sterilization drape (not shown) that is fitted over the mayo stand frame 18 and the tray 24 during a surgical procedure and into which the hook portion 80 comes into contact. In addition, the upper hook portion 80 is sufficiently low profile and thin such that it can be interposed between the frame and the mayo tray without interrupting flush and stable seating of the mayo tray 24 on the upper portion of the frame 18. The hook portion 80 is preferably fixedly coupled to the engagement portion 82, but may be removably coupled or may be articulable relative thereto. In a fixedly coupled relationship, a buttress 83 or other structural reinforcement may be fixed to and between the hook portion 80 and engagement portion 82 to provide additional strength and positional stability therebetween.

In an embodiment of the system, the engagement portion 82 include a pair of arms 84, 86 that are adapted to extend about a portion of the bottom 42 and the side walls 44, 46 at the proximal end portion 58 of the receptacle to thereby retain the receptacle. The arms 84, 86 define a tapered opening that accommodates the tapered shape of the receptacle 34 such that the receptacle is receivable therebetween and retained without passage completely therethrough. Particularly, at least a portion of each arm 84, 86 has a corresponding shape to the exterior of the proximal end portion 58 of the receptacle 34, and more particularly each arm preferably includes a tapered corner wall portion 88 against which the tapered corner wall portion 64 of the receptacle 34 engages in contact. Optionally, the arms 84, 86 may be resilient and sized or otherwise adapted to provide a clamping force to stably hold the receptacle 34 at the locations indicated, or at another location about the sides walls of the receptacle. As another alternative, other structure can be provided to preferably releasably retain the receptacle 34 relative to the support 32 in the configuration discussed above and below.

The engagement portion 82 may also be provided with a cover portion 90 which is optionally provided with a perimeter lip 92. When the engagement portion 82 and receptacle 34 are coupled relative to each other, the cover portion 90 extends over the open top 76 of the receptacle 34 to (i) provide a protective cover thereover, (ii) increase laterally rigidity to the side walls 44, 46 of the receptacle thereby reinforcing the receptacle construction, and (iii) form a stop 94 along distal portion of the lip 92 for the distal end 48 of the receptacle once the receptacle is fully inserted into the engagement portion 82. The lip 92 may also be provided with a mating structure 96 in the form, e.g., of a ridge or channel that engages relative to the catch 74 in the distal end 48 of the receptacle 34 to facilitate retaining the receptacle in the support, particularly once the receptacle is loaded with the surgical instrument. All of these features allow the receptacle to be made of less rigid, and preferably less expensive, materials than the receptacle support, as well as facilitate certain manufacturing processes of the receptacle, as noted above.

In accord with another aspect of the system, the support is provided with a cord holder 98 adapted to retain a portion of the instrument's flexible cord 110 to and at the side of the engagement portion of the support; i.e., particularly external of the receptacle and away from the surgical field. In the exemplar embodiment shown, the cord holder 98 is a preferably resilient C-shaped hook that extends from one of the arms 84. Alternatively or additionally, the cord holder may be provided to both of the arms 84, 86, the hook 80, the cover portion 90, and/or the perimeter lip 92. The cord holder 98 defines a new path for the flexible cord 110 of corded surgical instruments 100 in an operating room. The flexible cord 110 extends from under the patient operating bed 26, under the mayo stand 10, up under the mayo stand, and is coupled to the frame 18 of the mayo stand by the support system 30. The cord holder 98 keeps the flexible cord 110 out of the sterile field and prevents the cord from tangling. Thus, by attaching the flexible cord to the frame of the mayo stand, a most preferred, beneficial and safest route for the cord is provided. Referring to FIG. 2, it is appreciated that the cord also may be attached to the frame 18 of the mayo stand using a dedicated cord clip 500 which attached directly to the frame, e.g., in the same manner as the upper hook portion 80 of the support 30, and can support a cord 110a relative to the frame 18, under the frame, and out of the sterile field.

In accord with a preferred aspect of the system, the support 32 is configured to stably receive and suspend the receptacle 34 below the frame 18 of the mayo stand, preferably at least partially within footprint (i.e., under the perimeter) of the frame 18 of the mayo stand, and preferably at an oblique angle relative to the top of the frame of the mayo stand with the mouth of the receptacle oriented higher along the longitudinal axis A_{L} of the receptacle than its distal end. It is preferred that the longitudinal axis A_{L} extend at an angle between 1° and 89° from horizontal, and more preferably between 30° and 60° from horizontal, and most preferably between 30° and 45° from horizontal for a combination of best ergonomics and gravity feed of the instruments into the receptacle. The bottom 42 of the receptacle preferably extends parallel to the longitudinal axis A_{L}, and thus is oriented at the same relative angle. This permits the surgical instrument 100 to be gravity fed along the bottom 42 from the mouth 40 toward the distal end 48. As such, with the receptacle so held and oriented, the receptacle is adapted to receive, retain and orient the surgical instrument 100 for the surgeon in a convenient and accessible location during a surgical procedure.

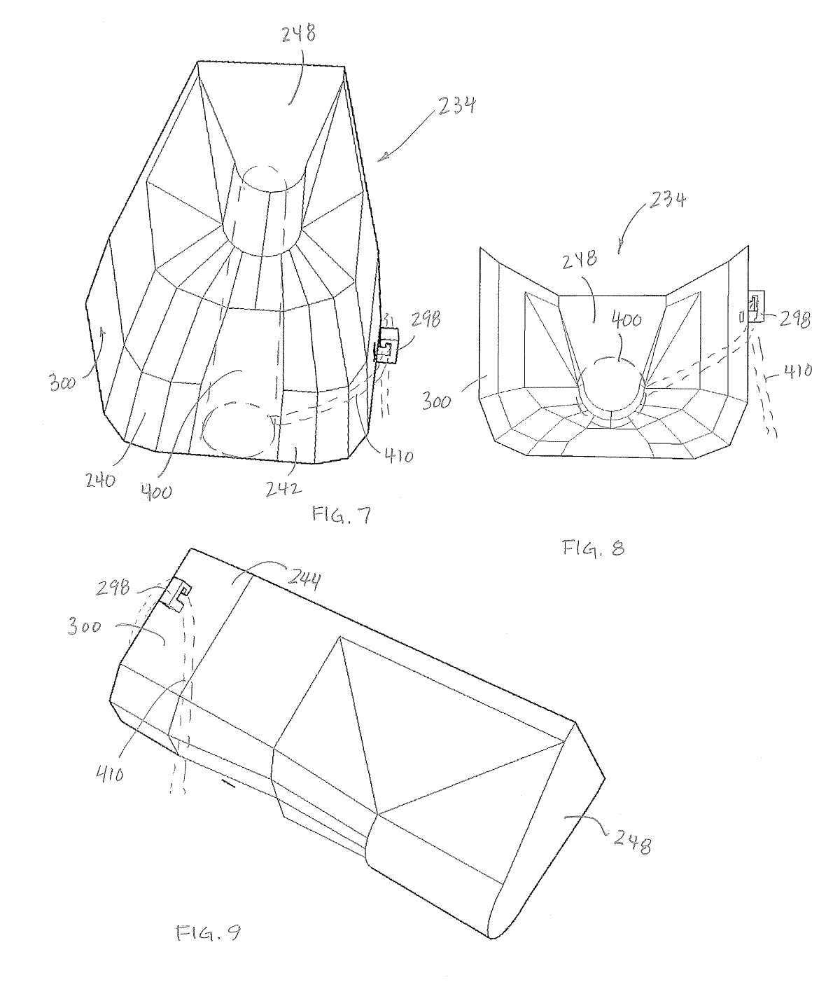

Turning now to FIGS. 7 through 9, another receptacle 234 is shown and is similar to receptacle 34 and engageable relative to the support in the same manner as previously described. Unless otherwise indicated, all prior descriptions and variations of receptacle 34 shall apply to receptacle 234 (with like parts incremented by 200). However, in distinction from receptacle 34, receptacle 234 has: (i) no slot extending in its bottom 242 and which is open to its mouth 240, (ii) a closed end 248, and (iii) preferably a relatively longer length. This combination of features allows the receptacle 234 to stably receive and accommodate a surgical instrument 400 that does not have a large handle portion (in comparison to instrument 100) and which may have a length that does not require extension through the distal end 248 of the receptacle. By way of example only, the receptacle is suitable for receiving a Bovie® electrocautery instrument and like devices. The receptacle 234 may optionally be provided with a proximal extension 300 to increase its length. The proximal extension 300 preferably extends proximal to where the engagement arms 84, 86 of the support 30 engages the receptacle (see FIG. 2). An exemplar length of the receptacle 234 is preferably in the range of four to eight inches, and an exemplar width is preferably in the range of 1.5 to 4 (at the mouth) to 1 to 2 inches (at the distal end). Such extension 300 is generally in a U-shape. The receptacle 234 may also be directly provided with a cord holder 298 adapted to retain a portion of an instrument's flexible cord 410 to and at the side wall 244 of the receptacle and away from the surgical field. The cord holder 298 is preferably mounted or formed at the proximal extension 300. Similarly tray 34 also may be directly provided with such a cord holder 98a mounted or formed on the side wall 44 (or 46) (FIGS. 4 through 6, broken lines).

Referring to FIGS. 3 and 9, the support 32 is a universal type design adapted to releasably receive each of at least receptacles 34 and 234. In accord with one preferred aspect of the system, the support 30 is constructed of materials permitting it to be re-usable and re-sterilizable. Preferred materials for the support 30 include sterilizable materials including metal and plastic. Most preferred, the support is constructed from stainless steel. In accord with another preferred aspect of the system, the receptacles 34, 234 are constructed of a different material from the support and are intended as disposable single-use items. Preferred materials for the receptacles include sterilizable materials including plastic. A preferred plastic includes polypropylene. It is appreciated however that both the support 30 and receptacles 34, 234 may be manufactured from metal, and preferably then be re-usable and re-sterilizable, or that both the support and receptacles may be intended as a single use disposable devices, and manufactured from appropriate materials including, but not limited to, plastic. It is also recognized that the support may be an integrated construct with a receptacle such that they are permanently assembled and/or formed together.

It should be further appreciated that all the figures of the system together comprise a kit that includes a plurality of receptacles 34, 234, each of which can be used with the support 30. Such a kit allows a preferably re-usable support 30 to be provided for mounting relative to the mayo stand, and different types of disposable receptacles to be provided for mounting to the support depending on the procedure. Alternately or additionally, the kit may be provided entirely or reusable or disposable components, but permit a single type of support be constructed that can couple with multiple types of receptacles to thereby facilitate inventory control.

There have been described and illustrated herein several embodiments of a mayo stand instrument support system. While particular embodiments of the invention have been described, it is not intended that the invention be limited thereto, as it is intended that the invention be as broad in scope as the art will allow and that the specification be read likewise. Thus, while particular shapes and dimensions of an instrument receptacle have been disclosed, it will be appreciated that other shapes and dimensions may be used as well. Also, while a preferred orientation of the longitudinal axis of the receptacle is stated, for purposes of gravity feeding and retaining instruments, the described system also includes a receptacle orientated such that its longitudinal axis extends in either a fully vertical or fully horizontal orientation. It will therefore be appreciated by those skilled in the art that yet other modifications could be made to the provided invention without deviating from its scope as claimed.

1. An instrument support system for supporting a surgical instrument relative to a mayo stand, the mayo stand having an upright support and a frame extending about a perimeter which defines a footprint, the support system comprising:
a) a receptacle having a bottom on which the instrument is supported, sides to laterally retain the instrument, an open proximal end through which to receive the instrument, and an opposite distal end having an opening through which to allow passage of a distal portion of the instrument, the receptacle defining a longitudinal axis; and
b) a support including a first portion that couples to the receptacle, and a second portion that removably couples the first portion to the frame of the mayo stand so that the support suspends the receptacle below the frame and at least partially within the footprint of the frame.

2. The instrument support system of claim 1, wherein:
the support suspends the receptacle at an oblique angle relative to the top surface of the frame such that the open proximal end of the receptacle is oriented higher along the longitudinal axis of the receptacle than the distal end of the receptacle.

3. The instrument support system of claim 1, wherein:
the support includes a cord holder configured to support a flexible cord member of the surgical instrument external of the receptacle.

4. The instrument support system of claim 3, wherein:
the receptacle and the support are removably couplable relative to each other.

5. The instrument support system of claim 1, wherein:
the bottom of the receptacle includes a slot extending to the proximal end of the receptacle to allows a portion of a handle of the instrument to intersect a surface defined by the bottom of the receptacle.

6. The instrument support system of claim 1, wherein:
the receptacle has a tapered outer profile, and the first portion of the support defines a tapered opening into which the receptacle is received.

7. The instrument support system of claim 6, wherein:
the first portion of the support includes a pair of arms that extend about the sides of the receptacle.

8. The instrument support system of claim 7, wherein:
the receptacle has a completely open top, and the first portion defines a cover for at least a portion of the receptacle.

9. The instrument support system of claim 8, wherein:
the first portion includes a rim about the cover, the rim providing lateral support to the receptacle.

10. The instrument support system of claim 1, wherein:
the first portion of the support couples to a proximal portion of the receptacle.

11. The instrument support system of claim 1, wherein:
the second portion of the support includes a hook sized to stably clip over the frame, the hook having a curved blunt end.

12. The instrument support system of claim 1, wherein:
the receptacle and the support are each made of different materials.

13. An instrument support system for supporting a surgical instrument relative to a mayo stand, the mayo stand having an upright support and a frame extending about a perimeter which defines a footprint, the support system comprising:
a) a receptacle having a bottom on which the instrument is supported, sides to laterally retain the instrument, an open proximal end through which to receive the instrument, and an opposite distal end, the receptacle defining a longitudinal axis; and
b) a support including a first portion that couples to the receptacle, and a second portion that removably couples the first portion to the frame of the mayo stand so that the support suspends the receptacle below the frame and at least partially within the footprint of the frame; and
c) at least one of the support and the receptacle includes a cord holder configured to support a flexible cord member of the surgical instrument relative to the one of the support and the receptacle.

14. The instrument support system of claim 13, wherein:
the cord holder is mounted to the receptacle.

15. The instrument support system of claim 13, wherein:
the receptacle and the support are adapted to be removably couplable relative to each other.

16. The instrument support system of claim 13, wherein:
the receptacle has a tapered outer profile, and the first portion defines a tapered opening into which the receptacle is received.

17. The instrument support system of claim 16, wherein:
the first portion of the support includes a pair of arms that extend about the sides of the receptacle.

18. The instrument support system of claim 17, wherein:
the receptacle has a completely open top, and the first portion defines a cover for at least a portion of the receptacle.

19. The instrument support system of claim 18, wherein:
the first portion includes a rim about the cover, the rim providing lateral support to the receptacle.

20. The instrument support system of claim 13, wherein:
the first portion of the support couples to a proximal portion of the receptacle.

21. The instrument support system of claim 13, wherein:
the receptacle and the support are each made of different materials.

22.-29. (canceled)
